(19) United States
(12) Patent Application Publication
(10) Pub. No.: US 2004/0220469 A1
(43) Pub. Date: Nov. 4, 2004

(54) Knee-foot coil with improved homogeneity

(76) Inventors: Jovan Jevtic, West Allis, WI (US); Derek Seeber, Wauwatosa, WI (US); Ashok Menon, Milwaukee, WI (US)

Correspondence Address: QUARLES & BRADY LLP, 411 E. WISCONSIN AVENUE, SUITE 2040, MILWAUKEE, WI, 53202-4497, US

(21) Appl. No.: 10/428,457

(22) Filed: May 2, 2003

(51) Int. Cl. A61B 5/055

(52) U.S. Cl. 600/422

(57) ABSTRACT

A knee-foot coil provides side coils covering both a side of a tubular foot support and a side of an attached toe chamber. Homogeneity in the signals from these loops is provided by a shunt, separating these side coils into portions with different sensitivities. Upper and lower coils provide for vertical sensitivity, the upper coil optionally surrounding the toe chamber.

### CROSS-REFERENCE TO RELATED APPLICATIONS

This application is a continuation-in-part of U.S. application Ser. No. ______ filed Mar. 3, 2003.

### STATEMENT REGARDING FEDERALLY SPONSORED RESEARCH OR DEVELOPMENT

--

### BACKGROUND OF THE INVENTION

The present invention relates to magnetic resonance imaging (MRI) and in particular local coils for use in transmitting radio frequency excitation signals and/or receiving magnetic resonance signals in magnetic resonance imaging.

Magnetic resonance imaging is used to generate medical diagnostic images by measuring faint radio frequency signals (magnetic resonance) emitted by atomic nuclei in tissue (for example, protons) after radio frequency stimulation of the tissue in the presence of a strong magnetic field.

The radio frequency stimulation may be applied, and the resulting magnetic resonance signal detected, using a “local coil” having one or more single turn conductive “loops” serving as antennas. The loops of the local coil are tuned to a narrow band, for example, 64 megahertz for a 1.5 Tesla field-strength magnetic field, and adapted to be placed near or on the patient to decrease the effects of external electrical noise on the detected magnetic resonance signal. The detected magnetic resonance signal may be conducted through one or more signal cables to the MRI machine for processing.

A local coil may incorporate multiple loops whose signals may be combined prior to being processed by the MRI machine. For example, in a quadrature type coil, perpendicular loops are combined with a 90° phase shift. Alternatively, the signals of the multiple loops may be conducted independently to the MRI machine to provide for the so-called “phased array” detection.

An important characteristic of a local coil is the homogeneity of its field strength, the latter defined as the coil's sensitivity to magnetic resonance signals when operated in a receive mode, and the strength of the coil's transmission of radio frequency excitation signals when operated in the transmit mode. Homogeneity is particularly important for certain MRI procedures such as fat saturation where too much or too little field strength may detrimentally affect the imaging process.

Field strength is a complex function of the design of the local coil and of the coil's interaction with the patient. Homogeneity is often a compromise with other desirable coil characteristics including signal-to-noise ratio and selection of a coil shape.

Desirably, a local coil is designed to conform closely to that volume of the patient with which the local coil will be used. In this regard, a patient's foot may be imaged with a local coil having a tubular chamber into which the foot is placed and a vertically oriented “chimney” for receiving the toes of the foot. The same coil may be used for knee imaging with the knee centered within the tubular chamber. A knee-foot coil of this design using a birdcage array of conductors is described in U.S. Pat. No. 5,277,183 issued Jan. 11, 1994 and assigned to the assignee of the present invention and hereby incorporated by reference.

An alternative conductor layout for such a coil might use one or more independent loops for obtaining signals. The shape of the coil form, however, is such as to place the loops, or portions of the loops, at varying distances from the foot, producing a coil that has poor homogeneity over the entire foot.

### BRIEF SUMMARY OF THE INVENTION

The present invention provides a foot coil using independent loops attached to the tubular form and perpendicular toe chamber so that at least one loop covers one side of both the tubular form and the toe chamber and a second loop encircles the toe chamber. Inhomogeneity in the side loop may be managed by placing a shunt across the loop to divide the current in the loop to create two loop portions, each with controllable field sensitivities. The portion of the loop covering the toes of the foot thus may be decreased in field sensitivity to provide more homogenous field coverage. Extension of the foot through the second loop allows the second loop to provide coverage of both the foot and toes.

Specifically, the present invention provides an MRI coil suitable for imaging a patient's foot, the coil having a tubular form extending along a first axis to receive a portion of the patient's leg there along and the patient's foot therein. A toe chamber extends perpendicularly to the first axis and from atop of the tubular form to receive toes of the patient's foot when the patient's foot is located in the tubular form. A conductive first loop has a first portion extending along a side of the tubular form and a second portion extending along the side of the toe chamber to provide sensitivity along a first axis in the tubular form and toe chamber. A conductive second loop extends along the top of the tubular form to provide sensitivity along a second axis substantially perpendicular to the first axis in the tubular form and toe chamber.

Thus it is one object of the invention to provide a simple coil structure for imaging a human foot that provides quadrature detection.

The first loop may include a shunt conductor dividing the first portion from the second portion and the first loop may be tuned to a resonant frequency so that the current flow at the resonant frequency within the first loop divides to be unequal in the first and second portions.

Thus it is another object of the invention to provide a simple loop antenna structure that may be controlled in field sensitivity to allow it to receive signals homogenously from both the toe region, and the ankle and heel region of the foot.

The amount of current flow may be a function of the area of the loops and their proximity to the foot.

Thus it is another object of the invention to provide greater flexibility in designing the physical aspects of the coil and, in particular, for allowing the tubular portion to be sized amply for ease of access of either the foot or the knee, while keeping the toe chamber compact, without significantly affecting coil homogeneity.

An additional third loop, having a first portion extending along a second side of the tubular form, and a second portion extending along a second side of the toe chamber, and positioned opposite the first loop, may also be employed and currents adjusted in this loop also using a shunt.

Thus it is another object of the invention to provide a Helmholtz configuration known to provide field uniformity therebetween together with the improved homogeneity from using the shunt.

A conductive fourth loop may extend along a bottom surface of the tubular form to oppose the second loop.

Thus it is another object of the invention to provide for both vertical and horizontal sensitivities such as may be used, for example, in quadrature combination to improve signal-to-noise ratio.

These particular objects and advantages may apply to only some embodiments falling within the claims and thus do not define the scope of the invention.

### BRIEF DESCRIPTION OF THE DRAWINGS

FIG. 1 is a perspective view of a simple loop having a conductive shunt per the present invention, wherein the loop is tuned to provide co-cyclic current flow such as decreases current flow at one end of the loop for reduced field sensitivity at that end;

FIG. 2 is a cross-sectional view of a head coil constructed of multiple simple loops similar to FIG. 1 showing increased proximity of a superior end of the loops to the patient as would normally produce an undesirable higher field strength which may be reduced by the shunt conductor of the present invention;

FIG. 3 is a perspective view of the head coil of FIG. 2 showing its domed top;

FIG. 4 is a simplified, schematic of the coil of FIG. 1 and of individual coils of FIGS. 2 and 3 showing the use of series capacitors for tuning the coil to resonance;

FIG. 5 is a perspective view of a knee-foot coil using the design principles described with respect to FIG. 1;

FIG. 6 is a side, elevational view of the coil of FIG. 5, in phantom, showing the conductor of a side loop and the positioning of a shunt to control sensitivities of the side loop in two loop portions, one near the ankle and one near the toes;

FIG. 7 is a schematic diagram of the coil of FIGS. 5 and 6 showing the division of current flow through the two loop portions;

FIG. 8 is a perspective simplified view of the coil structure of the coils of FIGS. 5 through 7 showing a combination of the signals from loops coil in quadrature orientation;

FIG. 9 is a figure similar to that of FIG. 5 showing an alternative embodiment of a knee-foot coil with six loops arrayed around the circumference of a cylindrical form; and

FIG. 10 is a figure similar to that of FIGS. 5 and 9 showing an alternative embodiment of a knee-foot coil with eight loops formed from a proximal and distal grouping of four loops, the loops of each grouping arrayed around the circumference of a cylindrical form.

### DETAILED DESCRIPTION OF THE PREFERRED EMBODIMENT

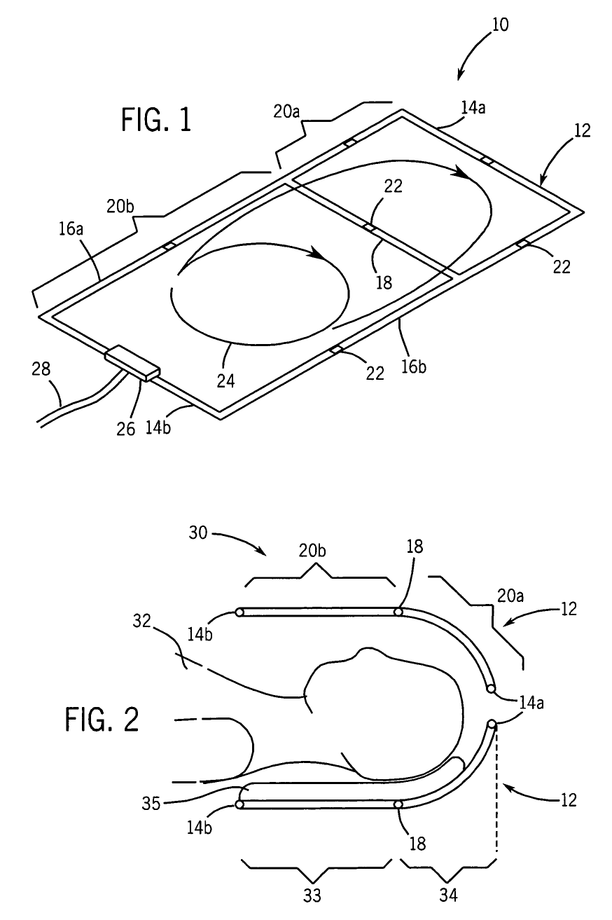

Referring now to FIG. 1, a local coil 10 for use with an MRI system, provides a series resonant electrical loop 12 and having first and second opposed end conductors 14a and 14b joined by opposed side conductors 16a and 16b. The form of the loop 12 as shown is rectangular, but the invention is not limited to this shape.

A shunt conductor 18 extending between the side conductors 16a and 16b generally parallel to the end conductors 14a and 14b, cuts the loop 12 into two loop portions 20a and 20b, loop portions 20a formed by end conductor 14a and shunt conductor 18 joined by portions of side conductors 16a and 16b and loop portions 20b formed by shunt conductor 18 and end conductor 14b joined by portions of side conductors 16a and 16b. Thus, the shunt conductor 18 is shared between the loop portions 20a and 20b.

A matching network 26 of a type well understood in the art may be connected to the local coil 10 at end conductor 14b to communicate through signal leads 28 to an MRI system (not shown) so that the local coil 10 may receive signals from the MRI system in a transmit mode and detect signals from the patient in a receive mode.

The local coil 10 is tuned into resonance through the use of capacitors 22 placed in series with the distributed inductances of the shunt conductor 18, end conductor 14a and 14b, and side conductors 16a and 16b. The tuning is such as to ensure that the resonant mode of the local coil 10 provides currents in loop portions 20a and 20b that are different by a desired amount. Generally, in the case of co-cyclic currents, current 24 passing through loop 20b in either direction splits at the junctures of the shunt conductor 18 and the side conductors 16a and 16b to pass partially through the shunt conductor 18 and partially through end conductor 14a so that the magnitude of the current 24 in loop 20b (being the measure of current in end conductor 14b) equals the magnitude of the current in the shunt conductor 18 summed with the magnitude of the current in the second loop portion 20a (being the measure of the current end conductor 14a). The currents need not be co-cyclic, however, for different tuning methods.

This splitting of the current 24 means that a radio-frequency (RF) excitation signal introduced into the local coil 10 by matching network 26 attached at end conductor 14b (during an MRI transmit cycle) will provide less current flow (and hence less field strength) at loop 20a than would be the case if the shunt conductor 18 were absent. Likewise during an MRI receive cycle, the magnetic resonance signal received by loop 20a will make a smaller contribution to the signal conducted from matching network 26 than would be the case if the shunt conductor 18 were absent.

Generally, the shunt conductor 18 may be varied in position along the length of side conductors 16a and 16b, with appropriate adjustment in the series capacitors 22, to change the point at which field strength is reduced. Multiple shunt conductors 18 (not shown) may be used to create several loop portions of reduced field strength.

As mentioned above, the loop 12 may operate in either a transmit or receive mode and when operating as a receive-only mode, local coil 10 may include passive or active de-coupling circuits of a type well known in the art.

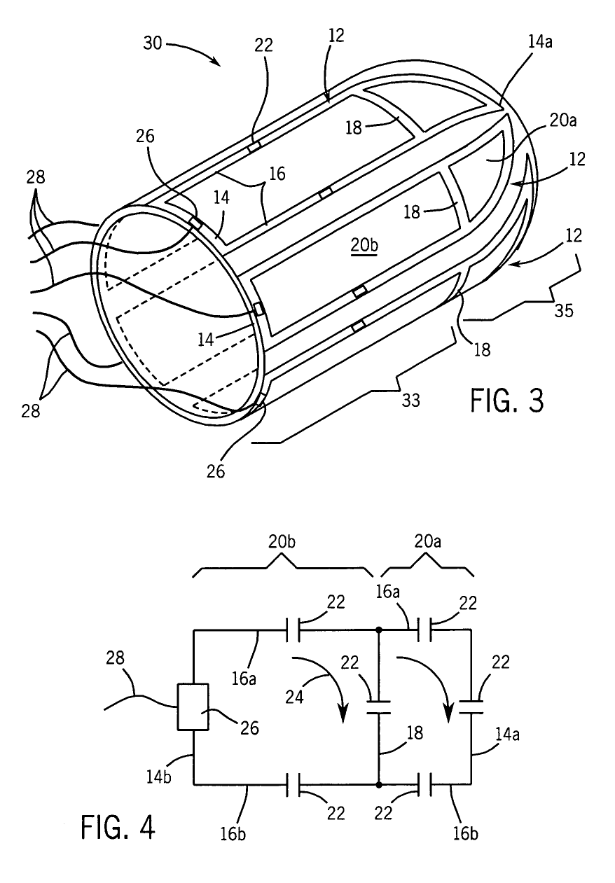

Referring now to FIGS. 2 and 3, an example application of the present invention provides a domed-top head coil 30 having a cylindrical tubular section 33 capped by a hollow hemispherical domed section 34 at its superior end. The inferior end of the domed-top head coil 30 is open to receive the head of a patient 32. The domed-top head coil 30 may include a patient support pillow 35 providing comfortable support of the patient's head and providing more uniformity in positioning of the patient within the volume of the domed-top head coil 30 so as to also enhance uniformity.

Loops 12, as described above, may be arrayed about the surface of the domed-top head coil 30 so that their side conductors 16 extend generally along the axis of the cylinder and the shunt conductors 18 of each loop 12 are positioned to be circumferential with respect to the cylinder generally at the interface between the cylindrical tubular sections 33 and the hemispherical domed section 34. Conductive ends 14a in this configuration are eliminated or reduced to extremely short segments so as to provide a tapering inward of the loop 12 as it approaches and covers the hemispherical domed section 34 accommodating the reduced circumference of that surface as one moves to its superior tip.

This tapering inward of the loop portions 20a of the loops 12 would normally be expected to cause increased field strength of loop portions 20a both because of their closer proximity to the patient 32 and because of their inward angulations. This increased field strength is offset, however, by the shunt conductor 18 which decreases the signal contributions to and by loop 20a as described above.

Each of the loops 12 in the domed-top head coil 30 may be separately connected by signal leads 28 and matching networks 26 to the MRI machine in a phased array mode of operation. Alternatively, each of the signal leads 28 may be joined to a combiner network properly phase shifting and adding these signals to produce one or more combination signals provided to the MRI machine. The signal leads 28 may be joined to follow along a grounding ring as taught in the U.S. patent application Ser. No. 10/227,072 filed Aug. 22, 2002, assigned to the assignee of the present invention and hereby incorporated by reference.

Referring now to FIG. 4 in the embodiment of the domed-top head coil 30, the shunt conductor 18 may be placed so as to create a ratio of areas between loop portion 20b and 20b of 2:1. In this situation, a current splitting through shunt conductor 18 versus end conductor 14a of approximately 1 to 0.6 as found suitable. Other ratios may also be appropriate for different configurations of coils other than that of FIG. 2 as will be understood to those of ordinary skill in the art.

It will be understood that the loops 12 may offer similar benefits in structures other than the domed-top head coil 30 but where portions of the patient anatomy may be closer or better received by portions of the loop or where the loop geometry would normally adversely affect field strength homogeneity in other ways.

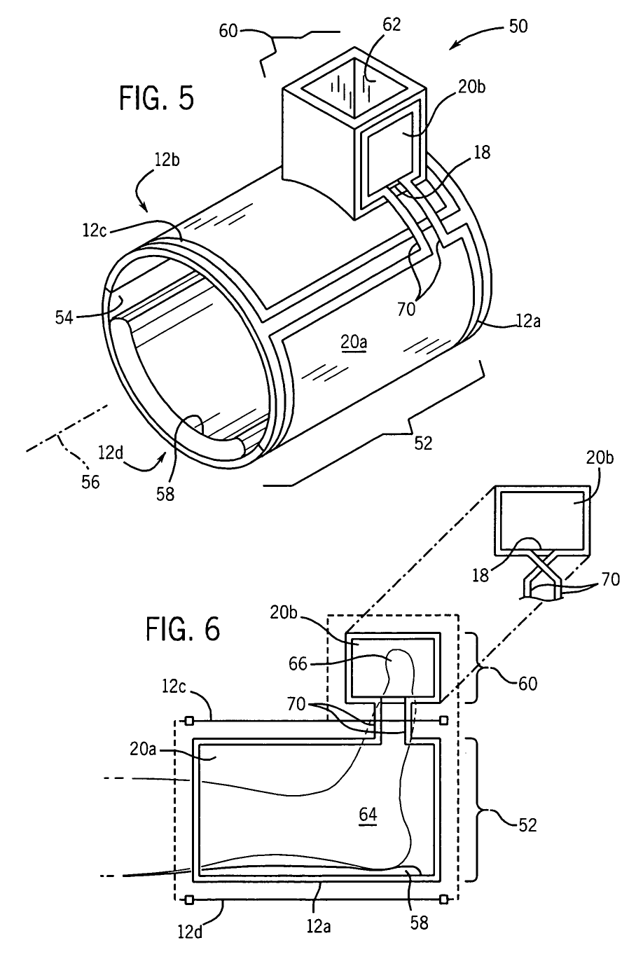

Referring now to FIG. 5, a knee-foot coil 50 using the above principles includes a tubular form 52 being generally cylindrical in shape and having a central lumen 54 extending along an axis 56 through which a patient's leg (shown in FIG. 6) may provide support for the back of a patient's leg.

A toe chamber 60 extends upward from the upper surface of the tubular form 52 and is generally a rectangular tube open at the top and bottom to define a vertical lumen 62. The terms “upper”, “top” and “vertical” and similar terms as used herein are references to the figure and/or a normal orientation of the coil and are not intended to be limitation to the invention which will work at different orientations. The lumen 62 of the toe chamber 60 communicates through an aperture in the top of the tubular form 52 (not visible) with the lumen 54.

Referring now to FIGS. 5 and 6, the back of the patient's foot may rest on the cushion 58 with the ankle 64 within the tubular form 52 and the patient's toes 66 extending upward into the toe chamber 60. A first loop 12a may be positioned to extend over both a right side of the tubular form 52 (per FIG. 5) and a right side of the toe chamber 60. The first loop 12a may be, for example, a layer of copper foil or other conductor adhered to the outer surface of the coil 50.

A shunt conductor 18 divides the loop 12a into a first and second portion 20a and 20b being on the sides of the tubular form 52 and toe chamber 60, respectively. Two bridging conductors 70 join the portion 20a and the portion 20b of the loop 12a.

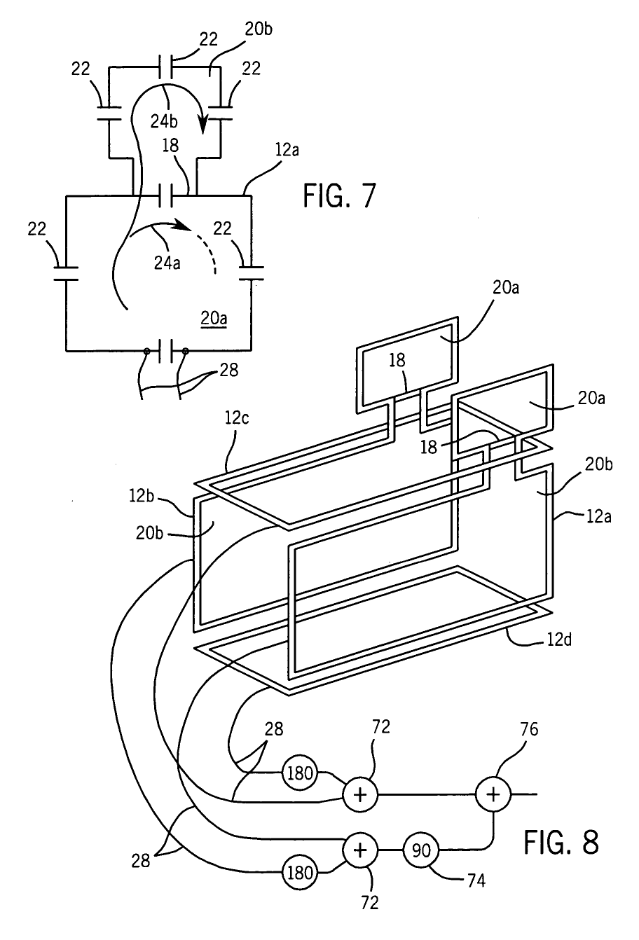

Referring now to FIG. 7 as before, loops portions 20a and 20b and shunt conductor 18 have series capacitors 22 which together with the distributed inductance of the conductor of loop portions 20a and 20b, tune the loop 12a into resonance at the resonant frequency of the MRI machine. Signal leads 28 passing to the MRI machine may attach to loop 20a, for example, across one of the series capacitors 22.

Ideally at resonance, current flow in loop 20a, indicated by arrow 24a, and current flow in loop 20b indicated by arrow 24b, are co-cyclic, that is, both either clockwise or simultaneously counterclockwise. If not, the loop portion 20a may be twisted with respect to the loop portion 20a to bring the current flows into a co-cyclic state. Specifically, the bridging conductors 70 may be crossed so as to reverse the sense of the loop portion 20b, as shown in FIG. 6 as an expanded fragment.

Generally, the area of the tubular form 52 encompassed by the first portion 20a of loop 12a is larger than the area of the toe chamber 60 encompassed by the second portion 20b of the loop 12a. For this reason, the impedance of the shunt conductor 18 is selected to reduce the current flow 24b with respect to the current flow 24a to offset what would otherwise be a greater field sensitivity of the loop portion 20b causing inhomogeneity of the coil 50.

Referring again to FIG. 5, symmetrically opposite from loop 12a, across a vertical plane through the coil 50, is loop 12b as may be also seen schematically in FIG. 8. Like coil 12a, coil 12b provides two portions, 20a and 20b, one portion being on the tubular form 52 and the other on the toe chamber 60.

Referring to FIG. 8, signals from signal leads 28 taken off of coils 12a and 12b may be combined by a network combiner 72 once they are given the proper phase so that their signals add for spins detected within the volume of the coil 50. The proper phase is obtained by effective phase shifting one of the signals from signal leads 28 from loops 12a and 12b, shown by combiner 72, which may for example, be a simple matching network that observes the proper polarity of the connections to those loops 12a and 12b.

Referring again to FIG. 5, loops 12a and 12b provide for horizontal sensitivity within the tubular form 52 and toe chamber 60. A vertical sensitivity is provided by a coil 12c positioned on the upper surface of the tubular form 52 to surround the toe chamber 60. A single loop 12c thus provides sensitivity to vertical fields produced by spins both in the toes 66 and ankle 64.

A corresponding loop 12d, visible in FIG. 6, is positioned symmetrically opposite from loop 12c, across a horizontal plane through the coil 50, on the tubular form 52 below the cushion 58. Loops 12c and 12d also include series tuning capacitors and are tuned to the frequency of the MRI machine.

Referring to FIG. 8, the signals from the loops 12c and 12d may also be combined by a combiner 72 after the proper polarity shifting, so that their signals add for horizontal fields.

The signals from the combiners 72 for the loop pair 12a and 12b may be shifted by ninety degree phase shifter 74 and combined with the signal from the coil pair 12c and 12d by combiner 76. The resulting combined quadrature signal provides improved signal-to-noise ratio arising from the fact that external noise will generally not observe a precise quadrature phasing, and thus will be reduced by the combination of the signals from this coil.

The present invention need not be limited to four loops 12 but may employ a greater number of loops 12, for example, six or eight that may operate together for transmitting an RF signal or receiving an NMR signal using standard phase shifting splitters and combiners.

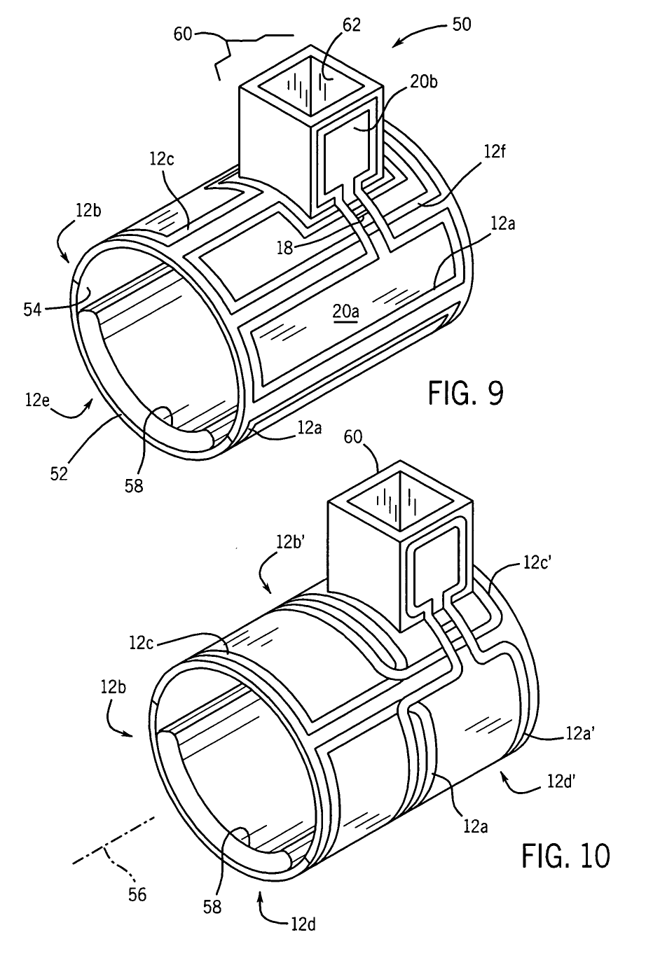

As shown in FIG. 9, side loops 12a and 12b and top and bottom loops 12c and 12d may be reduced in angular extent around the tubular form 52 from approximately 90 degrees, described above, to approximately 60 to accommodate two additional loops 12e and 12f for a total of six loops 12. These six loops 12 may be equally spaced in angle around axis 56 with loop 12f placed between loops 12a and 12c and loop 12e placed between loops 12b and 12d. With the necessary slight shifting of loop 12c, loop 12c may no longer encircle the toe chamber 60 but may provide an inward deviation in its conductor to accommodate the toe chamber 60 and to flank the toe chamber 60 with loop 12f. Loops 12a and 12b still include portions on both the side of the tubular form 52 and the side of the toe chamber 60 to provide a horizontal axis of sensitivity, while the loops 12c and 12f provide a vertical sensitivity in the toe chamber 60 and tubular form 52.

In an alternative embodiment shown in FIG. 10, side loops 12a and 12b and top and bottom loops 12c and 12d may be reduced in longitudinal extent along the axis of the tubular form 52 and moved toward a proximal end of the tubular form 52 away from the toe chamber 60. In this way, four more loops 12a′, 12b′, 12c′, and 12d′ can be added at the distal end of the tubular form toward the toe chamber 60. These loops 12a′, 12b′, 12c′, and 12d′ are aligned angularly with the loops 12a, 12b, 12c, and 12d and loop 12c′ encircles the toe chamber 60. Adjacent conductors of the pairs of loops (i.e., loop 12a and 12a′, loop 12b and 12b′, loop 12c and 12c′, loop 12e and 12e′) overlap to provide for decoupling as is understood in the art. This decoupling may be augmented with capacitive decoupling as required.

Loops 12a′ and 12b′ include portions on both the side of the tubular form 52 and the side of the toe chamber 60 to provide a horizontal axis of sensitivity, while the loops 12c′ and 12f′ provide a vertical sensitivity in the toe chamber 60 and tubular form 52.

It is specifically intended that the present invention not be limited to the embodiments and illustrations contained herein, but include modified forms of those embodiments including portions of the embodiments and combinations of elements of different embodiments as come within the scope of the following claims.

We claim:

1. An MRI coil suitable for imaging a patient's foot comprising:
a tubular form extending along a first axis to receive a portion of the patient's leg there along and patient's foot therein;
a toe chamber extending perpendicularly to the first axis and from a top of the tubular form for receiving toes of the patient's foot;
a conductive first loop having a first portion extending along a side of the tubular form and a second portion extending along the side of the toe chamber to provide sensitivity along a first axis in the tubular form and toe chamber; and
a conductive second loop extending along the top of the tubular form to provide sensitivity along a second axis substantially perpendicular to the first axis in the tubular form and toe chamber.

2. The MRI coil of claim 1 including a shunt conductor in the first loop dividing the first portion from the second portion and wherein the first loop is tuned to a resonant frequency and wherein current flow at the resonant frequency within the first loop divides so that the current flow in the first portion and the current flow in the second portion are unequal.

3. The MRI coil of claim 1 wherein an area circumscribed by the second portion is less than the area circumscribed by the first portion of the first loop and the shunt divides the current so that the current flow in the second portion is less than the current flow in the first portion of the first loop.

4. The MRI coil of claim 1 wherein the second portion is closer to the foot than the first portion of the first loop when the patient's foot is positioned in the MRI coil and the shunt divides the current so that the current flow in the second portion is less than the current flow in the first portion.

5. The MRI coil of claim 1 including a conductive third loop having a first portion extending along a second side of the tubular form and a second portion extending along a second side of the toe chamber, the third loop positioned opposite the first loop.

6. The MRI coil of claim 1 including a shunt conductor in the third loop dividing the first portion from the second portion and wherein the third loop is tuned to a resonant frequency and wherein current flow at the resonant frequency within the third loop divides so that the current flow in the first portion and the current flow in the second portion are unequal.

7. The MRI coil of claim 1 wherein the second loop encircles the toe chamber and further including a conductive fourth loop extending along a bottom surface of the tubular form opposite the second loop.

8. The MRI coil of claim 1 further including a matching network for producing a signal related to the current flow in the first portions of the first and second loop for transmission to an MRI machine.

9. The MRI coil of claim 1 further including:
a conductive third loop having a first portion extending along a second side of the tubular form and a second portion extending along a second side of the toe chamber, the third loop positioned opposite the first loop; and
a conductive fourth loop extending along a bottom surface of the tubular form opposite the second loop.

10. The MRI coil of claim 9 further including a quadrature combiner combining signals from the first and third loops with the signals from the second and fourth loops, the signals from the first and third loops shifted in phase by ninety degrees with respect to the signals from the second and fourth loops.

11. The MRI coil of claim 9 wherein each loop includes a decoupling circuitry decoupling the loop from radio frequencies transmitted by the MRI machine.

12. The MRI coil of claim 1 further including:
four additional conductive loops distributed about over the tubular form.

13. The MRI coil of claim 12 wherein the four additional conductive loops are distributed with the first and second loops evenly around the circumference of the tubular form.

14. The MRI coil of claim 12 wherein two of the loops are adjacent to opposite sides of the toe chamber without either of the two adjacent loops encircling the toe chamber.

15. The MRI coil of claim 1 further including:
six additional conductive loops distributed over the tubular form.

16. The MRI coil of claim 15 wherein the eight loops are arranged into proximal and distal grouping of four loops, the loops of each grouping arrayed in equal angle around the circumference of a cylindrical form.
